(19) United States
(12) Patent Application Publication
(10) Pub. No.: US 2006/0142227 A1
(43) Pub. Date: Jun. 29, 2006

(54) Amphiphylic peptide-PNA conjugates for the delivery of PNA through the blood brain barrier

(76) Inventors: Itschak Lamensdorf, Modiin (IL); Jehoshua Katzhendler, Jerusalem (IL)

Correspondence Address: PEARL COHEN ZEDEK, LLP, 1500 BROADWAY 12TH FLOOR, NEW YORK, NY, 10036, US

(21) Appl. No.: 10/962,669

(22) Filed: Oct. 13, 2004

Related U.S. Application Data
(60) Provisional application No. 60/510,139, filed on Oct. 14, 2003.

(51) Int. Cl. A61K 48/00; C07H 21/04; A61K 31/675

(52) U.S. Cl. 514/44; 514/81; 536/23.2; 544/81

(57) ABSTRACT

The invention provides molecules comprising a nucleic acid, a hydrophobic moiety and a positively charge moiety, useful in the delivery of a nucleic acid sequence across a cellular membrane. The invention further relates to the use of these molecules for the delivery of a nucleic acid sequence to the brain across the blood brain barrier for diagnostic and therapeutic applications.

### CROSS REFERENCE DATA

This Application claims the priority of U.S. Provisional Application No. 60/510,139 filed Oct. 14, 2003.

### FIELD OF THE INVENTION

The invention provides molecules comprising a nucleic acid, a hydrophobic moiety and a positively charge moiety, useful in the delivery of a nucleic acid sequence across a cellular membrane. The invention further relates to the use of these molecules for the delivery of a nucleic acid sequence to the brain across the blood brain barrier for diagnostic and therapeutic applications.

### BACKGROUND OF THE INVENTION

Antisense drugs are small complementary strands of DNA (oligonucleotides; ODNs) designed to bind to a specific sequence of nucleotides in the mRNA target, thus inhibiting production of the encoded protein. Antisense drugs work at early stages in the disease-causing process, are much more selective, easy to design, less complex and less expensive than do the traditional drugs. However, due to their low biomembrane permeability and their relatively rapid degradation oligonucleotides are generally considered to be of limited therapeutic value. To improve their therapeutic applications, the backbone of these antisense compounds has been chemically modified. The third generation of antisense chemistry is the polyamide (peptide) nucleic acid (PNA) surrogates. PNAs are the first successful substitutes of ODNs that have displayed equal or better binding affinity than natural DNA or RNA antisense-based drugs. PNAs are hydrophilic macromolecules and their administration required disruption of plasma membrane. Therefore, unmodified/naked PNA molecules pass poorly through the cell membrane and do not have useful therapeutic applications. In order to improve their cellular uptake PNAs were conjugated to delivery moieties such as positively charged peptide, receptor ligands or hydrophobic moiety.

Several properties of antisense-based drugs suggest that these compounds will have tremendous potential as future therapeutics for CNS disorders. As many of the proteins involved in the pathogenesis of CNS disorders are similar to other (healthy) vital proteins, and most if not all conventional drugs lack selectivity for the disease-target proteins a gene specific method is desirable. In addition, antisense-based drugs inhibit the production of encoded proteins, by acting at early stages in the disease-causing process. Since these compounds restrain the synthesis of the protein, their effect is long lasting and depends tightly upon the formation of new protein molecules. As most CNS disorders are chronic, the long-lasting activity of a drug should provide significant improvement in the patient's compliance, thus being therapeutically desirable.

Unfortunately, both ODNs and PNAs cannot cross the endothelial cellular membrane of the blood brain barrier (BBB) effectively, thus, preventing the possible use of these technologies in developing drugs for CNS disorders.

### SUMMARY OF THE INVENTION

In one embodiment, the invention provides a molecule represented by any one of the formulas I-IV:
[(H)_{q}—(N)ᵣ—(P)ₛ]ₓ I.
[(N)ᵣ—(H)_{q}—(P)ₛ]ₓ II.
[(P)ₛ—(H)_{q}—(N)ᵣ]ₓ III.
[(N)ᵣ,(P)ₛ—(H)_{q}]ₓ IV.
wherein N is a nucleic acid sequence in a length of 1-100 bases, H is a hydrophobic moiety and P is a positively charge moiety; and
wherein q is an integer of 1-20, r is an integer of 0-20, s is an integer of 1-25 and x is an integer of 1-20.

In one embodiment, the invention further provides a method for delivering a molecule across a cellular membrane.

In one embodiment, the invention further provides a method for delivering a nucleic acid sequence to the brain across the blood brain barrier.

### BRIEF DESCRIPTION OF THE DRAWINGS

The invention will be understood and appreciated more fully from the following detailed description taken in conjunction with the appended drawings in which:

FIG. 1 demonstrates fluorescence images of NMB cells and NMB cells incubated with fluorescein-labelled peptide-PNA conjugated or fluorescein-labelled unmodified PNA.

FIG. 2 demonstrates the uptake of conjugated PNAs into NMB cells

FIG. 3 demonstrates the uptake of conjugated PNAs into PC12 cells.

FIG. 4 demonstrates the uptake of conjugated PNAs into bEND3 cells in different incubation periods (a) and in different conjugated PNA concentrations (b).

### DETAILED DESCRIPTION OF THE INVENTION

In one embodiment, the invention provides a molecule represented by any one of the formulas I-IV:
[(H)_{q}—(N)ᵣ—(P)ₛ]ₓ V.
[(N)ᵣ—(H)_{q}—(P)ₛ]ₓ VI.
[(P)ₛ—(H)_{q}—(N)ᵣ]ₓ VII.
[(N)ᵣ—(P)ₛ—(H)_{q}]ₓ VIII.
wherein N is a nucleic acid sequence in a length of 1-100 bases, H is a hydrophobic moiety and P is a positively charge moiety; and
wherein q is an integer of 1-20, r is an integer of 0-20, s is an integer of 1-25 and x is an integer of 1-20.

In one embodiment of the invention, N is a nucleic acid sequence in a length of 1-100 bases. In another embodiment, N is a nucleic acid sequence in a length of 1-10 bases. In another embodiment, N is a nucleic acid sequence in a length of 1-20 bases. In another embodiment, N is a nucleic acid sequence in a length of 10-20 bases. In another embodiment, N is a nucleic acid sequence in a length of 20-30 bases. In another embodiment, N is a nucleic acid sequence in a length of 30-40 bases. In another embodiment, N is a nucleic acid sequence in a length of 40-50 bases. In another embodiment, N is a nucleic acid sequence in a length of 50-100 bases.

In one embodiment of the invention, q is an integer of 1-20. In another embodiment, q is an integer of 2-10. In another embodiment, q is an integer of 6-16. In another embodiment of the invention, q is 8. In another embodiment of the invention, q is 9.

In one embodiment of the invention, r is an integer of 0-20. In another embodiment, r is an integer of 1-10. In another embodiment, r is an integer of 10-20. In another embodiment, r is an integer of 2-5.

In one embodiment of the invention, s is an integer of 1-25. In another embodiment, s is an integer of 2-15. In another embodiment, s is an integer of 2-6. In another embodiment, s is 4.

In one embodiment of the invention, x is an integer of 1-20. In another embodiment, x is an integer of 2-15. In another embodiment, s is an integer of 5-10

In one embodiment of the invention, the nucleic acid sequence is a mRNA. In another embodiment of the invention, the nucleic acid sequence is a cDNA. In another embodiment the nucleic acid sequence is a DNA. In another embodiment of the invention, the nucleic acid sequence is a DNA analog. In another embodiment of the invention, the nucleic acid sequence is a PNA. In another embodiment the nucleic acid sequence is a PNA morpholino. In another embodiment of the invention, the nucleic acid sequence is an aminoethylprolyl (aep) PNA. In another embodiment of the invention, the nucleic acid sequence is a pyrrolidinyl PNA. In another embodiment of the invention, the nucleic acid sequence is an oligonucleotide. In another embodiment the nucleic acid sequence is an oligonucleotide analog. In another embodiment of the invention, the nucleic acid sequence is a ribozyme. In another embodiment of the invention, the nucleic acid sequence is an RNAi. In another embodiment of the invention, the nucleic acid sequence is a PNA.

In one embodiment of the invention, nucleic acid sequence is an antisense. In another embodiment of the invention, the nucleic acid sequence is an antigene. In another embodiment of the invention, the nucleic acid sequence is a decoy function. In one embodiment of the invention, the nucleic acid sequence is neutral. In another embodiment of the invention, the nucleic acid sequence is negatively charged. In one embodiment of the invention, nucleic acid sequence is in antisense orientation to an endogenous sequence.

The term “nucleotide” describes a subunit of DNA or RNA consisting of a nitrogenous base (adenine, guanine, thymine, or cytosine in DNA; adenine, guanine, uracil, or cytosine in RNA), a phosphate molecule, and a sugar molecule (deoxyribose in DNA and ribose in RNA). Thousands of nucleotides are linked to form a DNA or RNA molecule.

The term “oligonucleotide” describes a molecule usually composed of 25 or fewer nucleotides.

The term “antisense” describes a nucleic acid sequence that has a sequence exactly opposite to an mRNA molecule made by the body; binds to the mRNA molecule to prevent a protein from being made.

The term “peptide nucleic acids” (PNAs) refers to molecules that in certain respects are similar to oligonucleotide analogs however in other very important respects their structure is very different. In peptide nucleic acids, the deoxyribose phosphate backbone of oligonucleotides has been replaced with a backbone more akin to a peptide than a sugar phosphodiester. Each subunit has a naturally occurring or non-naturally occurring base attached to this backbone. A non-limiting example is a backbone constructed of repeating units of N-(2-aminoethyl)glycine or analogues thereof having a nucleobase attached thereto via a linker such as a carboxymethyl moiety or analogues thereof to the nitrogen atom of the glycine portion of the unit. The units are coupled together via amide bonds formed between the carboxyl group of the glycine moiety and the amine group of the aminoethyl moiety. The nucleobase can be one of the four common nucleobases of nucleic acids or they can include other natural or synthetic nucleobases. Due to the radical deviation from the deoxyribose backbone, these molecules were named peptide nucleic acids.

The term “antigene” refers to molecules, which bind to double-stranded DNA. Antigenes can enhance or inhibit gene expression in cells.

PNA combine peptide chemistry with nucleic acid sequence biology. In one embodiment, the invention provides a method for using peptide delivery technologies in the nucleic acid sequence field. In one embodiment of the invention, the PNA molecule is used as an antisense moiety, an antigene moiety or a gene modulator moiety, while in the another embodiment of the invention, the PNA molecule is used as an apolar peptide-like moiety in an amphiphlic brain vector.

In one embodiment, the invention provides a PNA-peptide conjugate whereas the PNA oligomer is used as an apolar moiety in the chimeric amphiphilic construct. In one embodiment of the invention, the molecule is composed from three sequent ional component: PNA oligomer, hydrophobic and a positively charged moieties. In one embodiment of the invention, the molecule is designed to have a positively charged moieties conjugated to the N or C terminals of hydrophobic modified PNA oligomer.

In one embodiment of the invention, the hydrophobic moiety is a nucleic acid. In another embodiment, the hydrophobic moiety is a nucleic acid analog. In another embodiment of the invention, the hydrophobic moiety is a hydrophobic peptide. In another embodiment of the invention the hydrophobic moiety is a lipid acid. In another embodiment of the invention, the hydrophobic moiety is a lipid molecules. In another embodiment of the invention, the hydrophobic moiety is octanol. In another embodiment of the invention, the hydrophobic moiety is cholesterol. In another embodiment of the invention, the hydrophobic moiety is a hydrophobic peptide protecting group. In another embodiment of the invention, the hydrophobic moiety is adamantine. In another embodiment of the invention, the hydrophobic moiety is pyrene. In another embodiment of the invention, the hydrophobic moiety is eicosenoic acid. In another embodiment of the invention, the hydrophobic moiety is a C₍₆₋₁₆₎ glyceride lipid. In another embodiment of the invention, the hydrophobic moiety is phenoxazine. In another embodiment of the invention, the hydrophobic moiety is a DMT group. In another embodiment of the invention, the hydrophobic moiety is cholenic acid. In another embodiment of the invention, the hydrophobic moiety is lithocholic acid. In another embodiment of the invention, the hydrophobic moiety is myristic acid. In another embodiment of the invention, the hydrophobic moiety is palmitic acid. In another embodiment of the invention, the hydrophobic moiety is a heptadecyl group. In another embodiment of the invention, the hydrophobic moiety is hexadecylglycerol. In another embodiment of the invention, the hydrophobic moiety is a geranyloxyhexyl group. In another embodiment of the invention, the hydrophobic moiety is hexadecylamine. In another embodiment of the invention, the hydrophobic moiety is dihydrotestosterone. In another embodiment of the invention, the hydrophobic moiety is 1-pyrene butyric acid. In another embodiment of the invention, the hydrophobic moiety is alkanoic acid. In another embodiment of the invention, the hydrophobic moiety is alkanol. In another embodiment of the invention, the hydrophobic moiety is and any derivatives of the above mentioned moieties. In one embodiment of the invention, the alkanoic acid is represented by the structure R—(CH₂)n—COOH, wherein n is an integer of 1-20 and R is a linear or branched alkyl. In another embodiment of the invention, n is an integer of 6-16. In one embodiment of the invention, the alkanol is represented by the structure R—(CH₂)n—OH, wherein n an integer of 1-20 and R is a linear or branched alkyl. In another embodiment of the invention, n is an integer of 6-16. In one embodiment of the invention, the lipid acid is undecanoic acid. In another embodiment of the invention, the lipid acid is docosahexanenonic acid. In one embodiment of the invention, the hydrophobic peptide protecting group is Fmoc. In another embodiment of the invention, the hydrophobic peptide protecting group is Tboc.

As contemplated herein, an “alkyl” group refers to a saturated aliphatic hydrocarbon, including straight-chain, branched-chain and cyclic alkyl groups. In an embodiment of the invention, the alkyl group has 1-4 carbons. In another embodiment, the alkyl group is a methyl group. In another embodiment, the alkyl group is an ethyl group. In another embodiment, the alkyl group is a propyl group. In another embodiment, the alkyl group is a butyl group. The alkyl group may be unsubstituted or substituted by one or more groups selected from halogen, hydroxy, alkoxy carbonyl, amido, alkylamido, dialkylamido, nitro, amino, alkylamino, dialkylamino, carboxyl, thio and thioalkyl.

In one embodiment of the invention, the positively charge moiety is a nucleic acid sequence or a nucleic acid analog. In another embodiment, the positively charge moiety is a positively charge peptide. In another embodiment, the positively charge moiety is a peptidomimetic. In another embodiment, the positively charge moiety is a polycations. In another embodiment, the positively charge moiety is 2-O-aminopropyl. In another embodiment, the positively charge moiety is 2-O-dimethylaminopropyl. In another embodiment, the positively charge moiety is 2-O-imidazolyl-ethyl. In another embodiment, the positively charge moiety is 2-O-aminoethylamino-oxyethyl. In another embodiment, the positively charge moiety is 2-dimethylaminoethyl-oxyethyl. In another embodiment, the positively charge moiety is and any derivative of the above mentioned moieties. In another embodiment, the positively charge moiety is arginine. In another embodiment, the positively charge moiety is D-arginine. In another embodiment, the positively charge moiety is polyarginine. In another embodiment, the positively charge moiety is polyamine. In another embodiment, the positively charge moiety is guanidine. In another embodiment, the polyamine is spermine. In another embodiment, the polyamine is spermidine. In another embodiment, the polyamine is putricine.

In one embodiment of the invention, the hydrophobic moiety, the nucleic acid sequence and the positively charge moiety are linked to each other directly via peptide bonds.

In one embodiment, the invention provides a molecule comprising undecanoic acid, a PNA and at least one arginine group. In another embodiment, the undecanoic acid is directly or indirectly linked, on one side to the PNA and on the other side to at least one arginine group. In another embodiment, the PNA is directly or indirectly linked, on one side to the undecanoic acid and on the other side to at least one arginine group.

In one embodiment, the invention provides a molecule comprising undecanoic acid, a PNA, morpholino or any neutral nucleic acid analogs and at least one arginine group. In another embodiment, the undecanoic acid is directly or indirectly linked, on one side to the PNA, morpholino or any neutral nucleic acid analogs and on the other side to at least one arginine group. In another embodiment, the PNA morpholino is directly or indirectly linked, on one side to the undecanoic acid and on the other side to at least one arginine group.

In one embodiment, the invention provides a molecule comprising undecanoic acid, an oligonucleotide and at least one arginine group. In another embodiment, the undecanoic acid is directly or indirectly linked, on one side to the oligonucleotide and on the other side to at least one arginine group. In another embodiment, the oligonucleotide is directly or indirectly linked, on one side to the undecanoic acid and on the other side to at least one arginine group. In one embodiment of the invention, the oligonucleotide is an amphiphilic oligonucleotide.

In one embodiment, the invention provides a molecule comprising a PNA, an antisense moiety and at least one arginine group. In another embodiment, the PNA is directly or indirectly linked, on one side to the antisense moiety and on the other side to at least one arginine group. In another embodiment, the antisense moiety is directly or indirectly linked, on one side to the PNA and on the other side to at least one arginine group.

In one embodiment, the invention provides a molecule comprising a PNA, an antisense moiety and at least one arginine group. In another embodiment, the PNA is directly or indirectly linked, on one side to the antisense moiety and on the other side to at least one arginine group. In another embodiment, the antisense moiety is directly or indirectly linked, on one side to the PNA and on the other side to at least one arginine group.

In one embodiment, the invention provides a molecule comprising an oligonucleotide, an antisense moiety and at least one arginine group. In another embodiment, the oligonucleotide is directly or indirectly linked, on one side to the antisense moiety and on the other side to at least one arginine group. In another embodiment, the antisense moiety is directly or indirectly linked, on one side to the oligonucleotide and on the other side to at least one arginine group. In one embodiment of the invention, the oligonucleotide is an amphiphilic oligonucleotide.

In one embodiment, the invention provides a molecule comprising undecanoic acid, a PNA and at least one polyamine group. In another embodiment, the undecanoic acid is directly or indirectly linked, on one side to the PNA and on the other side to at least one polyamine group. In another embodiment, the PNA is directly or indirectly linked, on one side to the undecanoic acid and on the other side to at least one polyamine group. In another embodiment, the polyamine is spermidine. In another embodiment, the polyamine is putricine.

In one embodiment, the invention provides a molecule comprising undecanoic acid, a PNA and at least one guanidinium group. In another embodiment, the undecanoic acid is directly or indirectly linked, on one side to the PNA and on the other side to at least one guanidinium group. In another embodiment, the PNA is directly or indirectly linked, on one side to the undecanoic acid and on the other side to at least one guanidinium group.

In one embodiment, the invention provides a molecule comprising docosahexanenonic acid (DHA)-PNA (1-20 bases)-(Arg)₄. In one embodiment, the invention provides a molecule comprising undecanoic acid —PNA (1-20 bases)-(Arg)₄. In one embodiment, the invention provides a molecule comprising PNA-DHA-(Arg)₄. In one embodiment, the invention provides a molecule comprising undecanoic acid-CCGCTCCG (SEQ ID NO. 1)-(Arg)₂₋₁₅. In one embodiment, the invention provides a molecule comprising docosahexanenonic acid-CAT GGT GGA CGT (SEQ ID NO. 2)-(Arg)₂₋₁₅. In one embodiment, the invention provides a molecule comprising undecanoic acid-CAT GGT GGA CGT (SEQ ID NO. 3)-(Arg)₂₋₁₅. In one embodiment, the invention provides a molecule comprising docosahexanenonic acid-CTT TCT CCT TTT CC (SEQ ID NO. 4)-(Arg)₂₋₁₅. In one embodiment, the invention provides a molecule comprising undecanoic acid —CTT TCT CCT TTT CC (SEQ ID NO. 4)-(Arg)₂₋₁₅. In one embodiment of the invention, the PNA is in a length of 5-10 bases.

In one embodiment, the invention provides a molecule comprising DHA (SEQ ID NO. 5)-PNA (0-20 bases)-(deoxynucleic guanidine-DNG)₁₋₂₀. In one embodiment, the invention provides a molecule comprising PNA (0-20 bases)-DHA-(DNG)₁₋₂₀ In one embodiment, the invention provides a molecule comprising DHA-PNA (0-20 bases)-(aminoethylprolyl (aep) PNA)₁₋₂₀ In one embodiment, the invention provides a molecule comprising PNA (0-20 bases)-DHA-(aepPNA)₁₋₂₀ In one embodiment, the invention provides a molecule comprising (Pyrrolidinyl PNA)₁₋₂₀-PNA (0-20 bases)-(aepPNA)₁₋₂₀ In one embodiment, the invention provides a molecule comprising PNA (0-20 bases)-(Pyrrolidinyl PNA)₁₋₂₀-(aepPNA)₁₋₂₀. In one embodiment of the invention, the PNA is in a length of 5-10 bases.

In one embodiment, the invention provides a molecule comprising TACTCATGGGCACACT (SEQ ID NO. 6)-FLFLRR (SEQ ID NO. 7). In one embodiment, the invention provides a molecule comprising (C)₈-TTT GCT CTT ACT CAT (SEQ ID NO. 8)-(D-Arg)₆. In one embodiment, the invention provides a molecule comprising PEG-(C)₈-TTT GCT CTT ACT CAT (SEQ ID NO. 8)-(D-Arg)₆. In one embodiment, the invention provides a molecule comprising PEG-TTT GCT CTT ACT CAT (SEQ ID NO. 8)-(C)₈— (D-Arg)₆. In one embodiment, the invention provides a molecule comprising TTT GCT CTT ACT CAT (SEQ ID No. 8)-PEG-(C)₈— (D-Arg)₆. In one embodiment, the invention provides a molecule comprising (Arg)₂-TACTCATGGGCACACT (SEQ ID NO. 9)-FLFLFL (SEQ ID NO. 10). In one embodiment, the invention provides a molecule comprising (Arg)₆-TACTCATGGGCACACT (SEQ ID NO. 9)-FLFLFL (SEQ ID NO. 10). In one embodiment, the invention provides a molecule comprising TTT GCT CTT ACT CAT (SEQ ID NO. 8)-(FL)₂₋₃ (SEQ ID NO. 11)-(Arg)₂₋₆. In one embodiment, the invention provides a molecule comprising (FL)₂₋₃ (SEQ ID NO. 11)-TTT GCT CTT ACT CAT (SEQ ID NO. 8)-(Arg)₂₋₆. In one embodiment, the invention provides a molecule comprising CHKKKKKKHC (SEQ ID NO. 12)-PNA (1-20 bases)-(FL)₂₋₃ (SEQ ID NO. 11). In one embodiment, the invention provides a molecule comprising CHKKKKKKHCC (SEQ ID NO. 13) PNA (1-20 bases)-(FL)₂₋₃ (SEQ ID NO. 11). In one embodiment, the invention provides a molecule comprising (CH₂)₉—CO-(CCGCTCCG) (SEQ ID NO. 15)-GRRRK (SEQ ID NO. 16). We note that (CH₂)₉—CO is a chemical structure. In one embodiment of the invention, the PNA is in a length of 5-10 bases.

In one embodiment, the invention provides a molecule comprising DHA-PNA (1-20 bases)-putricine. In one embodiment, the invention provides a molecule comprising PNA (1-20 bases)-DHA-putricine. In one embodiment, the invention provides a molecule comprising undecanoic acid-PNA (1-20 bases)-Putricine. In one embodiment, the invention provides a molecule comprising PNA (1-20 bases)-undecanoic acid-putricine. In one embodiment, the invention provides a molecule comprising undecanoic-PNA (1-20 bases)-spermine. In one embodiment, the invention provides a molecule comprising PNA (1-20 bases)-undecanoic acid-spermine. In one embodiment, the invention provides a molecule comprising undecanoic acid -PNA (1-20 bases)-spermidine. In one embodiment, the invention provides a molecule comprising PNA (1-20 bases)-undecanoic acid-spermidine. In one embodiment of the invention, the PNA is in a length of 5-10 bases.

In one embodiment, the invention provides a molecule comprising Fmoc-PNA(1-20 bases)-putricine. In one embodiment, the invention provides a molecule comprising. TBoc-PNA (1-20 bases)-putricine. In one embodiment, the invention provides a molecule comprising docosahexanenonic acid-PNA (1-20 bases)-Diethylenetriamine. In one embodiment, the invention provides a molecule comprising docosahexanenonic acid-PNA (1-20 bases)-polyethylenimine. In one embodiment, the invention provides a molecule comprising allyl substituted PNA (1-20 bases)-polyethylenimine. In one embodiment, the invention provides a molecule comprising chloro and/or bromo-halogenated PNA (1-20 bases)-polyethylenimine. In one embodiment, the invention provides a molecule comprising allyl substituted PNA (1-20 bases)-(Arg/D-Arg)₂₋₁₀. By Arg/D-Arg) it is meant, either arginine or the D-isomer of arginine (D-Arg), in any combination thereof. In one embodiment, the invention provides a molecule comprising chloro and/or bromo-halogenated substituted PNA (1-20 bases)-(Arg/D-Arg)₂₋₁₀. In one embodiment of the invention, the PNA is in a length of 5-10 bases. In one embodiment of the invention, the number of Arg/D-Arg groups is 2-6. In another embodiment, the number of Arg/D-Arg groups is 4.

In one embodiment of the invention, the molecule further comprising a linker moiety linking between the hydrophobic moiety, the nucleic acid sequence and the positively charge moiety. In another embodiment, the linker moiety is polyethylene glycol (PEG). In another embodiment, the molecular weight of said PEG is in the range of 2000-40,000. In another embodiment, the linker moiety is a disulfide. In another embodiment, the linker moiety is an amide. In another embodiment, the linker moiety is an amine. In another embodiment, the linker moiety is an oxyamine. In another embodiment, the linker moiety is an oxyimine. In another embodiment, the linker moiety is a morpholine. In another embodiment, the linker moiety is a thioether. In another embodiment, the linker moiety is thiourea sulfonamide. In another embodiment, the linker moiety is an ether. In another embodiment, the linker moiety is an ester. In another embodiment, the linker moiety is a carbonate. In another embodiment, the linker moiety is a carbamate. In another embodiment, the linker moiety is guanidine. In another embodiment, the linker moiety is avidin. In another embodiment, the linker moiety is strepavidin. In another embodiment, the linker moiety is biotin. In another embodiment, the linker moiety is praline. In another embodiment, the linker moiety is lysine. In another embodiment, the linker moiety is cysteine.

In one embodiment of the invention, the liable linker or peptide bond to polyethylene glycol conjugated to the molecule improves phramacokinetic properties and overcomes possible side effects induced by the amphiphilic PNA. In one embodiment of the invention, the linker is conjugated to the molecule via a known technology.

In one embodiment, the invention provides a molecule comprising CHKKKKKKHCC (SEQ ID. No. 17)-PNA (1-20 bases)-(FL)₂₋₃ (SEQ ID NO. 11). In one embodiment, the invention provides a molecule comprising CHKKKKKKHCC (SEQ ID. No. 17)-PNA (1-20 bases)-avidin/strepavidin-biotin-(FL)₂₋₃ (SEQ ID NO. 11). In one embodiment, the invention provides a molecule comprising CHKKKKKKHCC (SEQ ID. No. 17) PNA (1-20 bases)-(FL)₂₋₃ (SEQ ID NO. 11). In one embodiment, the invention provides a molecule comprising CHKKKKKKHCC (SEQ ID. No. 17) PNA (1-20 bases)-(FL)₂₋₃ (SEQ ID NO. 11). In one embodiment, the invention provides a molecule comprising (Arg/D-Arg)₂₋₆-PK (SEQ ID NO. 20)-CGC TGG GC (SEQ ID NO. 19) CC(FL)₂₋₃ (SEQ ID NO. 18). In one embodiment, the invention provides a molecule comprising (Arg/D-Arg)₂ 6-CGC TGG GC (SEQ ID NO. 20) CC(FL)₂₋₃ (SEQ ID NO. 18). In one embodiment, the invention provides a molecule comprising (Arg)₂₋₆-PEG-CGC TGG GC (SEQ ID NO. 20) CC(FL)₂₋₃ (SEQ ID NO. 18). In one embodiment, the invention provides a molecule comprising (Arg/D-Arg)₂₋₆-PEG-C—C (SEQ ID NO. 21)-CGC TGG GC (SEQ ID NO. 20)-CC(FL)₂₋₃ (SEQ ID NO. 18). In one embodiment of the invention, the PNA is in a length of 5-10 bases. In one embodiment of the invention, the number of Arg/D-Arg groups is 2-6. In another embodiment, the number of Arg/D-Arg groups is 4. In one embodiment, the invention the molecular weight of said polyethylene glycol is in the range of 2000-40,000.

In one embodiment, the invention further provides a composition comprising as an active ingredient an effective amount of one or more molecules according to claim 1, together with one or more pharmaceutically acceptable excipients or adjuvants. In one embodiment of the invention, the composition is formulated for oral or parenteral administration. In another embodiment, the composition is formulated as uncoated tablets, coated tablets, pills, capsules, powder or suspension. In another embodiment, the composition is formulated for intravenous administration. In another embodiment, the composition is formulated intranasal administration. In another embodiment, the composition is formulated administration via aerosols. In another embodiment, the composition is formulated for transdermal administration. In another embodiment, the composition is formulated in an ointment, cream or gel form. In another embodiment, the compositions of the invention are formulated in a liquid dosage form. Examples of suitable liquid dosage forms include solutions or suspensions in water, pharmaceutically acceptable fats and oils, alcohols or other organic solvents, including esters, emulsions, syrups or elixirs, solutions and/or suspensions.

Suitable excipients and carriers can be solid or liquid and the type is generally chosen based on the type of administration being used. Liposomes may also be used to deliver the composition. Examples of suitable solid carriers include lactose, sucrose, gelatin and agar. Oral dosage forms may contain suitable binders, lubricants, diluents, disintegrating agents, coloring agents, flavoring agents, flow-inducing agents, and melting agents. Liquid dosage forms may contain, for example, suitable solvents, preservatives, emulsifying agents, suspending agents, diluents, sweeteners, thickeners, and melting agents parenteral and intravenous forms should also include minerals and other materials to make them compatible with the type of injection or delivery system chosen.

In one embodiment, the invention provides a method for the synthesis of a molecule according to the invention.

In one embodiment, the invention further provides a method for delivering a nucleic acid sequence across a cellular membrane comprising the step of applying to a cell an effective amount of one or more molecules according to the invention. In one embodiment of the invention, the cell is an endothelial cell. In another embodiment, the cell is a neuronal cell. In another embodiment, the cell is a glial cell. In another embodiment, the cell is a muscle cell.

In one embodiment, the invention provides a method for the improved delivery of PNAs into mammalian cells. In one embodiment, the invention provides an amphiphilic PNA chimeric moiety with improved neuronal, endothelial delivery properties.

In one embodiment, the invention further provides a method for intracellular targeting of a nucleic acid sequence to an intracellular organelle comprising the step of applying to a cell an effective amount of one or more molecules according to the invention. In one embodiment of the invention, charge distribution, length of the apolar nucleic acid chain and hydrophobicity can affect sub-cellular compartization. In one embodiment, the invention provides a method for intracellular targeting of a nucleic acid sequence to an intracellular organelle comprising the step of applying to a cell an effective amount of one or more molecules of the invention, wherein the molecules crosse the nuclear membrane.

In one embodiment, the invention further provides a method for delivering a nucleic acid sequence to the brain across the blood brain barrier, said method comprising the step of administering to a subject an effective amount of one or more molecules according to the invention. In another embodiment, the invention provides a method for delivering a nucleic acid sequence to the brain across the blood brain barrier, said method comprising the step of administering to a subject a composition according to the invention.

In one embodiment of the invention, polyarginine oligomers are used to improve BBB penetration of PNA-based constructs In another embodiment, arginine guanido groups are used to improve BBB penetration of PNA-based constructs. In one embodiment of the invention, the increased brain uptake through the BBB is via guanidine basic amino acid transporters. In another embodiment, the increased brain uptake is the result of augmented AMT as a result of increased permeability surface due to the presence of positive charge.

In one embodiment of the invention, polyamines are used to improve BBB penetration of PNA-based constructs. In another embodiment, polyamine is putrescine. In another embodiment, polyamine is sperimidine. In another embodiment, polyamine is putrescine. In another embodiment, polyamine is spermine. In one embodiment of the invention, the increased brain uptakethrough the BBB is via polyamine transporters. In another embodiment, the increased brain uptake is the result of augmented AMT as a result of increased permeability surface due to the presence of positive charge.

In one embodiment, the invention further provides a method for delivering a gene across the blood brain barrier for expression in the brain, said method said method comprising administering to a subject an effective amount of one or more molecules according to the invention. In another embodiment, the invention provides a method for delivering a gene across the blood brain barrier for expression in the brain, said method said method comprising administering to a subject one a composition according to the invention.

In one embodiment, the invention further provides a method for modulating gene expression, said method said method comprising administering to a subject an effective amount of one or more molecules according to the invention. In another embodiment, the invention provides a method for modulating gene expression, said method said method comprising administering to a subject one a composition according to the invention.

In one embodiment, the invention provides a kit comprising an effective amount of one or more molecules according to the invention. In another embodiment, the kit allows gene labeling. In another embodiment, the kit further comprising labeling and/or reaction buffers. In another embodiment, the molecule is conjugated to a fluorescent label, a calorimetric label, a radiolabel label or a chemical label.

In one embodiment, the invention further provides a method for the treatment, prevention and control of a disease, said method comprising administering to a subject an effective amount of one or more molecules according to the invention. In another embodiment, the invention provides a method for the treatment, prevention and control of a disease, said method comprising administering to a subject a composition according to the invention. In one embodiment of the invention, the disease is a central nervous system related disease.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In case of conflict, the present specification, including definitions, will control.

This invention is further illustrated in the Experimental Details section, which follows. This section is set forth to aid in an understanding of the invention but is not intended to, and should not be construed to, limit in any way the invention as set forth in the claims that follow thereafter.

### EXPERIMENTAL DETAILS SECTION

Methods

Synthesis:

PNA and Peptide Synthesis.

Oligomers were made on a Chemspeed Automatic Synthesizer on 5 micromole scale applying double coupling of fluorenylmethoxycarbonyl (Fmoc) protected nucleobases monomers or amino-acids (AA) with a ratio of 1:3 each coupling cycle. Resin used was either wang resin (0.57 mmole/g) or TGA resin 9 Novasyn, 0.15 mmole/g) already loaded with amino acid. Total reaction volume was 250 microliter and coupling reagent utilized was either BOP or HBTU in the presence of diisopropylethylamine and 2,6-lutidine. PNAs or PNAs combined with peptides or/and with 11-amino undecanoic acid were deprotected and removed from the resin by trifluoro acetic acid 1 ml containing triethylsilyl (2 h). Product was percipitate with cold diethylether 14 ml collected and subjected to RP-HPLC on a 10 micron vydac column at 50° C. Gradient 10% to 90% acetonitril (CAN) in 25 min, rt˜10 min. Analysis: Mass-spectra: MALDI-TOF

Cell Culture:

As an in vitro models for neurons and for BBB we used the human neuroblastoma NMB cell line the rat catecholaminergic cell line PC 12 and the mice brain-derived endothelial cell line bEND3.

NMB cells were adapted to grow in Dulbecco modified Eagle medium (DMEM) with 10% Hyclone calf serum. PC12 cells were grown in DMEM, 10% horse serum and 5% calf serum. bEND3 cells were grown in DMEM, %10 calf serum supplemented with 2 mM glutamine. Cells were maintained in a 10% CO₂ humidified incubator at 37° C. Cells were routinely sub-cultured every four to five days. When cells (5×10³) were plated in 96 wells, dishes were pretreated with poly-L-ornithine.

Uptake Experiments:

Uptake of PNA and conjugated PNA was determined as previously published [4,9] by measurement of fluorescence retained in cells following incubation with fluorescein-labelled peptide-PNA conjugated or fluorescein-labelled unmodified PNA. The molecules were incubated with NMB. After incubation, the cells were extensively washed with ice-cold PBS followed by acid wash (1.0 M NaCl/0.4 M NaOAc, PH 3.3). PC12 or bEND3 for different time period. A procedure that was found to removed both noninternalized oligomer and dead cells. After the final wash intra-cellular fluorescence was determined by fluoremeter (FLUOstar BMG Labtechnologies).

### EXAMPLES

### Example 1

Fluorescence Microscopy

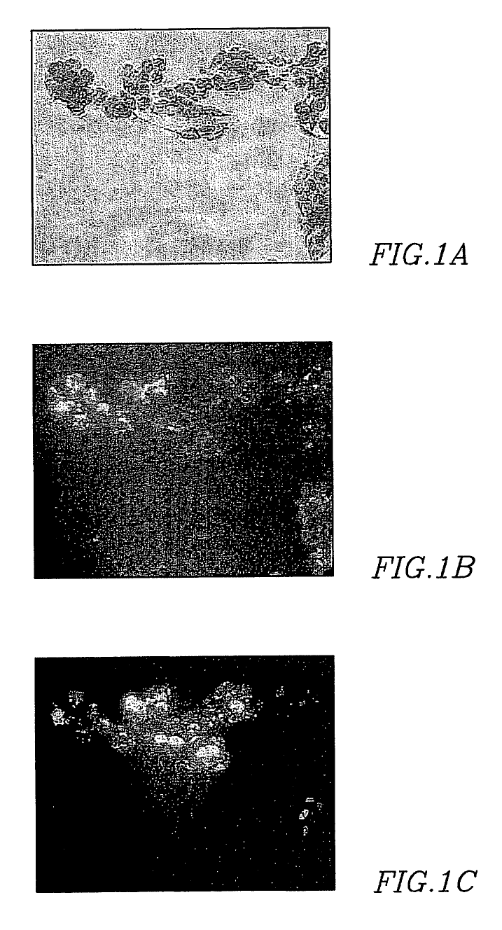

To test neuronal compartization and cellular distribution of unmodified PNA. The human neuronal cell line NMB cells were seeded on a poly-L-ornithine coated 35 mm dish. 24 hours following seeding the cell culture medium was replaced with DMEM containing 1 μM PNA. Cells were incubated with FITC labeled PNA and propidium iodide (PI) for nucleolus staining for 4 hours. Following incubation cells were washed 3 times with PBS and medium was replaced with fresh DMEM. Bright field and UV images were taken using Olympus BH-2 microscope. Images were digitized using the Power PC i.view 32 image analysis software (FIG. 1). As can be seen neuronal cell line are taking up unmodified PNA in a punctuated manner indicating that unmodified PNA are being tramped in the endosomal, lysosomal system.

### Example 2

NMB Cells

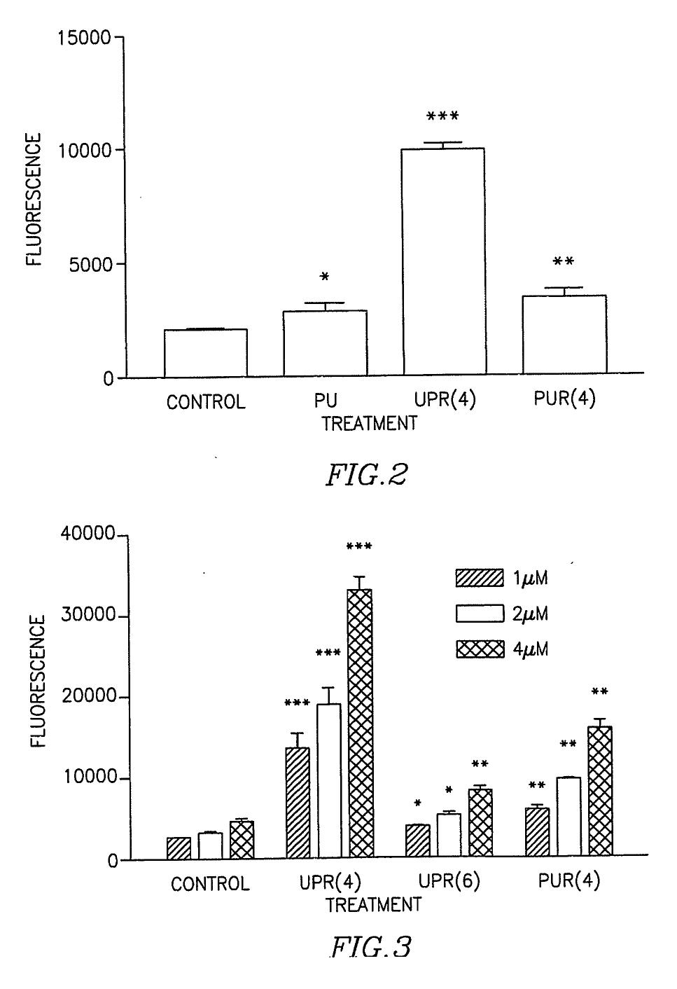

To test the effect of amphilic structure composition uptake of conjugated PNAs into the human neuronal cell line NMB was determined. Cells were seeded on 96 well dishes coated with poly-1-ornithine. Day after seeding medium was replaced with fresh medium without serum containing one micromolar PNAs. Following one-hour incubation medium was removed and cells were washed three times with acid wash solution and fluorescent determined (FIG. 2). P represents PNA, U represents undecanoic acid; R represents arginine; and (n) the number of arginine groups. The control group represents cells treated with PNA. Adding hydrophobic moiety increases PNA uptake. However, addition of amphiphylic moiety further increase PNA uptake. The highest intracellular content could be achieved when the PNA sequence was used as a neutral spacer between the hydrophobic and the positively charge components.

### Example 3

PC12 Cells

To test dose response and to optimize amphiphylic composition several modified PNAs were tested on PC12 cell line. Uptake of conjugated PNAs into the neuronal cell line PC12. Cells were seeded on 96 well dishes coated with poly-1-lysine. Day after seeding medium was replaced with fresh medium without serum containing different concentrations (1-4 μM) of PNAs. Following one-hour incubation medium was removed and cells were washed three times with acid wash solution and fluorescent determined (FIG. 3). P, U and R(n) are defined above. PNA uptake increase in a dose dependent manner. Amphiphilic modifications markedly enhanced PNA uptake. Changing the number of arginine residues affected modified PNA uptake as increasing the number of arginine from 4 to 6 monomers decreases neuronal uptake.

### Example 4

bEND3 Cells

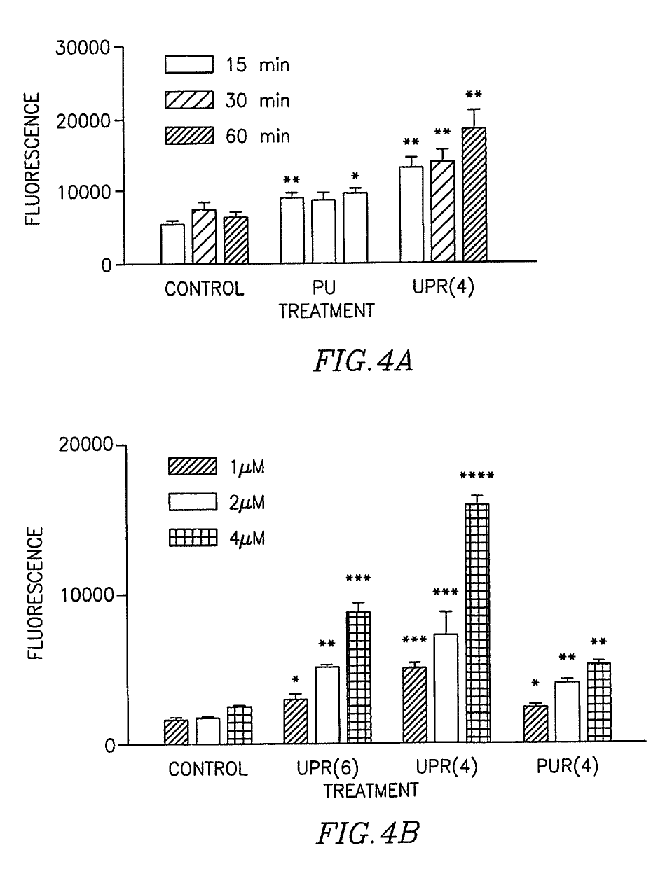

The BEND3 cell line (a murine cell line model of BBB) was used to test time and dose response dependency of modified PNA (Amphiphilic based modifications). Uptake of conjugated PNAs into the mice endothelial brain cell line bEND3. Cells were seeded on 96 well dishes coated with poly-1-ornithine. Day after seeding medium was replaced with fresh medium without serum containing one micromolar PNAs. Cells were incubated for different time period of 15 to 60 minutes. Following incubation medium was removed and cells were washed three times with acid wash solution with acid wash solution and fluorescent determined (FIG. 4a), P, U and R(n) are defined above. As can be seen UPR(4) had a higher uptake than the control or the PU indicating that amphipilic modification can increase penetration through the BBB.

Cells were also incubated with different concentrations of conjugated PNAs. Following incubation medium was removed and cells were washed three times with acid wash solution with acid wash solution and fluorescent determined (FIG. 4b). P, U and R(n) are defined above. In an excellent agreement with the neuronal cell line finding, also in the BBB model UPR(4) composition was found to be the most potent conjugation.

It will be appreciated that the invention is not limited by what has been described hereinabove and that numerous modifications, all of which fall within the scope of the invention, exist. Rather the scope of the invention is defined by the claims that follow:

What we claim is:

1. A molecule represented by any one of the formulas I-IV:
[(H)_{q}—(N)ᵣ—(P)ₛ]ₓ I.
[(N)ᵣ—(H)_{q}—(P)ₛ]ₓ II.
[(P)ₛ—(H)_{q}—(N)ᵣ]ₓ III.
[(N)ᵣ,(P)ₛ—(H)_{q}]ₓ IV.
wherein N is a nucleic acid sequence in a length of 1-100 bases, H is a hydrophobic moiety and P is a positively charge moiety; and
wherein q is an integer of 1-20, r is an integer of 0-20, s is an integer of 1-25 and x is an integer of 1-20.

2. The molecule according to claim 1, wherein said nucleic acid sequence is a mRNA, a cDNA, a DNA, a DNA analog, a polyamide nucleic acid (PNA), a PNA morpholino, an aminoethylprolyl (aep) PNA, a pyrrolidinyl PNA, an oligonucleotide, an oligonucleotide analog, a ribozyme or an RNAi.

3. The molecule according to claim 1, wherein said nucleic acid sequence is a PNA.

4. The molecule according to claim 2, wherein said nucleic acid sequence is an antisense, an antigene or a decoy function.

5. The molecule according to claim 1, wherein said nucleic acid sequence is neutral or negatively charged.

6. The molecule according to claim 1, wherein said hydrophobic moiety is a nucleic acid.

7. The molecule according to claim 1, wherein said hydrophobic moiety is hydrophobic peptides, lipid acid, lipid molecules, octanol, cholesterol, hydrophobic peptide protecting group, adamantine, pyrene, eicosenoic acid, C₍₆₋₁₆₎ glyceride lipid, phenoxazine, DMT group, cholenic acid, lithocholic acid, myristic acid, palmitic acid, heptadecyl group, hexadecylglycerol, geranyloxyhexyl group, hexadecylamine, dihydrotestosterone, 1-pyrene butyric acid, alkanoic acid, alkanol or any derivatives thereof.

8. The molecule according to claim 7, wherein said alkanoic acid is represented by the structure R—(CH₂)n—COOH, wherein n=1-20 and R is a linear or branched alkyl.

9. The molecule according to claim 7, wherein said alkanol is represented by the structure R—(CH₂)n—OH, wherein n=1-20 and R is a linear or branched alkyl.

10. The molecule according to claim 7, wherein said lipid acid is undecanoic acid and/or docosahexanenonic acid.

11. The molecule according to claim 7, wherein said hydrophobic peptide protecting group is Fmoc or Thoc.

12. The molecule according to claim 1, wherein said positively charge moiety is a nucleic acid.

13. The molecule according to claim 1, wherein said positively charge moiety is positively charge peptide, peptidomimetic, polycations, 2-O-aminopropyl, 2-O-dimethylaminopropyl, 2-O-imidazolyl-ethyl, 2-O-aminoethylamino-oxyethyl, 2-dimethylaminoethyl-oxyethyl or any derivative thereof.

14. The molecule according to claim 1, wherein said positively charge moiety comprises at least one group of arginine, polyamin and/or guanidine.

15. The molecule according to claim 14, wherein said polyamine is spermine, spermidine or putricine.

16. The molecule according to claim 1, wherein said hydrophobic moiety, said nucleic acid sequence and said positively charge moiety are linked to each other directly via peptide bonds.

17. The molecule according to claim 1, further comprising a linker moiety linking between said hydrophobic moiety, said nucleic acid sequence and said positively charge moiety.

18. The molecule according to claim 17, wherein said linker moiety is polyethylene glycol, disulfide, amide, amine, oxyamine, oxyimine, morpholine, thioether, thiourea sulfonamide, ether, ester, carbonate, carbamate, avidin, strepavidin, biotin, praline, lysine, cysteine, guanidine or any combination thereof.

19. The molecule according to claim 18, wherein the molecular weight of said polyethylene glycol is in the range of 2000-40,000.

20. A composition comprising as an active ingredient an effective amount of one or more molecules according to claim 1, together with one or more pharmaceutically acceptable excipients or adjuvants.

21. The composition according to claim 20, formulated for oral or parenteral or intravenous, transdermal, intranasal administration.

22. A method for delivering a nucleic acid sequence across a cellular membrane comprising the step of applying to a cell an effective amount of one or more molecules according to claim 1.

23. The method according to claim 22, wherein said cell is an endothelial cell, neuronal cell and glial cell.

24. A method for intracellular targeting of a nucleic acid sequence to an intracellular organelle comprising the step of applying to a cell an effective amount of one or more molecules according to claim 1.

25. A method for intracellular targeting of a nucleic acid sequence to an intracellular organelle comprising the step of applying to a cell an effective amount of one or more molecules according to claim 1, wherein said molecule crosses the nuclear membrane.

26. A method for delivering a nucleic acid sequence to the brain across the blood brain barrier, said method comprising the step of administering to a subject an effective amount of one or more molecules according to claim 1.


SEQUENCE LISTING (15 sequences)

SEQ ID NO: 1 - DNA, 8 bp, Homo sapiens
ccgctccg

SEQ ID NO: 2 - DNA, 12 bp, Homo sapiens
catggtggacgt

SEQ ID NO: 3 - DNA, 14 bp, Homo sapiens
ctttctccttttcc

SEQ ID NO: 4 - DNA, 16 bp, Homo sapiens
tactcatgggcacact

SEQ ID NO: 5 - protein, 6 aa, Homo sapiens
FLFLRR

SEQ ID NO: 6 - DNA, 15 bp, Homo sapiens
tttgctcttactcat

SEQ ID NO: 7 - DNA, 16 bp, Homo sapiens
tactcatgggcacact

SEQ ID NO: 8 - protein, 6 aa, Homo sapiens
FLFLFL

SEQ ID NO: 9 - protein, 10 aa, Homo sapiens
CHKKKKKKHC

SEQ ID NO: 10 - DNA, 8 bp, Homo sapiens
ccgctccg

SEQ ID NO: 11 - protein, 5 aa, Homo sapiens
GRRRK

SEQ ID NO: 12 - protein, 2 aa, Homo sapiens
PK

SEQ ID NO: 13 - DNA, 8 bp, Homo sapiens
cgctgggc

SEQ ID NO: 14 - protein, 6 aa, Homo sapiens
CCFLFL

SEQ ID NO: 15 - protein, 8 aa, Homo sapiens
CCFLFLFL